(19) United States
(12) Patent Application Publication
(10) Pub. No.: US 2026/0248644 A1
(43) Pub. Date: Aug. 27, 2026

(54) NOVEL ORAL COOLING DEVICE

(71) Applicant: SITISOLV AB, Kristianstad (SE)

(72) Inventors: David Wensbo Posaric, Lund (SE); Jenny Svensson, Tygelsjö (SE)

(21) Appl. No.: 19/167,439

(22) PCT Filed: Mar. 19, 2024
(86) PCT No.: PCT/SE2024/050246

§ 371 Date: Sep. 22, 2025

(30) Foreign Application Priority Data
Mar. 22, 2023 (SE) 2330133-6

(51) Int. Cl. A61F 7/12; A61F 7/00; A61F 7/02; A61F 7/10

(52) U.S. Cl. CPC A61F 7/12; A61F 7/02; A61F 7/10; A61F 2007/0017; A61F 2007/0087; A61F 2007/0219; A61F 2007/022; A61F 2007/0246; A61F 2007/0257; A61F 2007/108

(57) ABSTRACT

The present invention discloses an oral cooling device (1000) for user controlled local cooling of the oral cavity. It includes a distal region (100), adapted for placement within the oral cavity and carrying a heat transfer permeable outer layer (110), a proximal region (200), adapted for being gripped by the user and carrying a heat transfer restrictive outer layer (220), an internal heat transfer element (300) and an internal heat buffer (250). Upon use, heat is transferred from the oral cavity to the internal heat buffer (250) via a heat collecting region (310) and a heat emitting region (311) of the internal heat transfer element (300). Respectfully submitted,

### TECHNICAL FIELD

The present invention generally relates to a cooling of tissue, in particular to devices enabling local cooling of soft tissue within the oral cavity.

### BACKGROUND

There is a great medical need to alleviate thirst among patients in the immediate postoperative period, in patients undergoing intensive or palliative care and in elderly patients.

A vast number of physiological processes are strongly correlated to temperature. For example, fever, being a common bodily response e.g. to infection, is characterized in an increase in temperature above normal body temperature, believed to benefit the thereto related immune response. Another example is local cooling of a bodily part, which is often practiced for the reduction of associated inflammation and/or pain. Peripheral nerve signaling constitute one route through which a particular physiological response is suppressed or activated by a change in temperature.

Thirst is a response to the internal sensing of blood osmolarity being outside the limits of an optimally hydrated body. Thirst is, however, not quenched primarily by the return of such osmolarity to within limits, but rather as a result of sensory cues coupled to ongoing water consumption, e.g. from the oropharynx. Temperature appears to be one specific oropharyngeal mechanism for the detection of ongoing water consumption, as cold liquids inhibit subfornical organ thirst neurons to a higher degree as compared to hot liquids (D. E. Leib et al., Curr Biol., 2016, 26(24), 1260-1265). Emerging recognition of a physiological “thirst circuit”, and its dependance on low temperatures per se for quenching thirst, has been brought to wide attention by public media reviewing research progress (see for example J. Webb, BBC News, Aug. 4, 2016; https: //www. bbc. com/news/science-environment-36966275).

Accordingly, the most common methods used today to alleviate thirst among patients are all based on the provision of liquids that stimulate cold sensing neurons, either by having a low temperature, by comprising a pharmaceutically active compound that activates such neurons or by a combination of both. The latter include, for example, the use of mentholated popsicles (M. F. Conchon et al., J. Perianesth Nurs., 2021, 36(3), 262-267). Disadvantages of the provision of oral liquid include, for example, the associated medical hazard when the patient is cognitively impaired, has difficulties swallowing or the medical situation requires the patient to be slightly dehydrated. Disadvantages of oral administration of a pharmaceutically active compound that activates cold sensing neurons include, for example, potential adverse effects, e.g. such systemic effects, e.g. in an already medically challenged and weak patient.

US 2021/0177705 A1 discloses a pacifier for cooling of oral tissue, comprising an outer shell forming an outer chamber retaining a first cooling medium and, positioned therein, an inner shell forming an inner chamber retaining a second cooling medium. Disadvantages of this technical solution include, for example, the volumetric restriction of the combined cooling media, i.e. the first and the second cooling medium, to reside within the part of the pacifier intended for being inside the mouth when used. The effective time of use is thus restricted accordingly. Furthermore, the minimal size of the effective cooling area is correlated to that effective time, whereby the shortest acceptable effective time dictates the possible minimum size of the cooling area.

JP2008018199 (A) discloses a dental cooling device for cooling within the oral cavity. It comprises a tubular contact part being integrated with a bag-shaped coolant storage part. Upon use after an initial refrigeration step, heat is transferred from the oral cavity via the contact part and a liquid cooling agent to the coolant storage part. Disadvantages of this device include, for example, a risk of potentially hazardous leakage of liquid cooling agent into the oral cavity, should the contact part be punctured, e.g. by material fatigue in combination with biting on it.

US 2020/0069459 A1 discloses a mouthpiece for cooling of oral tissue, comprising an external chamber for storing a cooling medium having a freezing temperature below 0 degree Celsius and a bladder positioned therein containing a solution having a freezing point above the freezing point of the cooling medium. Disadvantages of this device include, for example, a necessity to enable a continuous or intermittent flow of the cooling medium to achieve optimal heat transfer from the oral cavity to the solution of the bladder.

Furthermore, there is a risk of potentially hazardous leakage of cooling medium into the oral cavity, should the mouthpiece be punctured, e.g. by material fatigue in combination with biting on it.

It would be advantageous with an improved oral cooling device that allows for avoiding at least some of the above mentioned disadvantages and associated problems.

### SUMMARY

It is an object of the invention, considering the disadvantages mentioned above, to provide an oral cooling device which allows for cooling within the oral cavity without simultaneously releasing any liquid or pharmaceutically active compound.

It is another object of the invention, considering the disadvantages mentioned above, to provide an oral cooling device which allows for patient controlled cooling within the oral cavity.

It is yet another object of the invention, considering the disadvantages mentioned above, to provide an oral cooling device with maximized effective use time.

It is further yet another object of the invention, considering the disadvantages mentioned above, to provide an oral cooling device with improved control of the effective cooling temperature.

Accordingly, examples of the present invention preferably seek to mitigate, alleviate or eliminate one or more deficiencies, disadvantages or issues in the art, such as the above-identified, singly or in any combination by providing an oral cooling device according to the appended patent claims.

According to a first aspect, there is provided an oral cooling device for user controlled local cooling of the oral cavity, having a distal region adapted for placement within the oral cavity, and a proximal region adapted for being gripped by a hand or at least two fingers of the user. The distal region is mechanically arranged in a spatially restricted relationship relative the proximal region such that a user induced movement of the latter results in a corresponding movement of the former. The outer surface of the distal region is at least partly constituted by a heat transfer permeable outer layer being in direct or indirect contact with a heat collecting region of an internal heat transfer element located in the inner volume of the distal region, for heat transfer from the oral cavity to the heat collecting region. The outer surface of the proximal region is constituted, fully or partly to an effective or acceptable degree, by a heat transfer restrictive outer layer enclosing an internal heat buffer and a heat emitting region of the internal heat transfer element in the inner volume of the proximal region, for the prevention of heat transfer from the surrounding to the internal heat buffer and to the heat emitting region. The heat emitting region and the internal heat buffer are joined or in direct contact with each other and adapted to maximize heat transfer between each other, by the former being heat sink shaped. The heat collecting region and the heat emitting region are joined or in direct contact with each other to together form the internal heat transfer element and adapted to maximize heat transfer between each other, optionally by the internal heat transfer element containing more than 1 weight-% of a metal or metal oxide, for heat transfer from the oral cavity to the internal heat buffer. The internal heat buffer is an aqueous salt solution with a melting point in the range of −20 to −1 °C.

According to a second aspect, the oral cooling device of the first aspect may be used for the prevention, reduction or amelioration of thirst.

According to a third aspect, a method for cooling an area within the human oral cavity or for reduction or amelioration of thirst is provided. The method comprising the consecutive steps of (i) cooling an oral cooling device of the first aspect to provide a cold oral cooling device of the first aspect, followed by (ii) cooling an area within the human oral cavity using the cold oral cooling device.

Further examples of the invention are defined in the dependent claims.

Some examples of the disclosure provide for self-controlled cooling within one's own oral cavity without assistance from another person.

Some examples of the disclosure provide for assisted or self-controlled amelioration of thirst without oral administration of water or a pharmacologically active compound.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which examples of the invention are capable of will be apparent and elucidated from the following description of examples of the present invention, reference being made to the accompanying drawings, in which:

FIG. 1 is a perspective view from the front/side of the oral cooling device 1000, showing a distal region 100 protruding from a proximal region 200, which proximal region 200 is being gripped by the hand of a user, such as e.g. a patient or caregiver, according to one embodiment of the invention;

FIGS. 2A and 2B is a view from the side and from the back, respectively, of the oral cooling device 1000 of FIG. 1, showing a cut-in-shaped outer geometry of the proximal region 200 adapted for gripping, according to one embodiment of the invention;

FIG. 3 is a cut-through view from the side of the oral cooling device 1000 of FIG. 1, showing a heat transfer permeable outer layer 110 of the distal region 100, being in direct contact with an underlying heat collecting region 310 of an internal heat transfer element 300, which internal heat transfer element 300 extends into the proximal region 200 in the form of a heat sink shaped heat emitting region 311 being in direct contact with an internal heat buffer 250 within the inner void of a surrounding heat transfer restrictive outer layer 220, according to one embodiment of the invention;

FIG. 4A shows an oral cooling device 1000 having a distorted cylinder proximally expanding rounded proximal region 200, adapted for holding with one hand, and a distal region 100 consisting of two separate protruding extensions, adapted for the simultaneous cooling of two opposing areas in the oral cavity, according to one embodiment of the invention;

FIG. 4B shows an oral cooling device 1000 having a donut-shaped proximal region 200, adapted for holding with one hand, and a tablet-shaped distal region 100, according to one embodiment of the invention; and

FIG. 4C shows an oral cooling device 1000 having a donut-shaped proximal region 200, adapted for holding with one hand, and a popsicle-shaped distal region 100, according to one embodiment of the invention.

FIG. 5 is a perspective view from the front/side of an internal heat transfer element 300 adapted for an oral cooling device 1000 having a spherically or pseudo-spherically shaped proximal region 200 (not shown), showing a heat collecting region 310 extending into a heat sink shaped heat emitting region 311 having a pseudo-spherically outer form and comprising fin-like extensions, in the proximal-distal direction, extending radially in a tree-growing fashion, in the radial direction, according to one embodiment of the invention.

FIG. 6 shows the internal heat transfer element 300 of FIG. 5 in heat-transfer operation at a sustained constant temperature gradient, i.e. at temperature gradient equilibrium, in which heat, illustrated in type and direction as arrows, is transported from the surrounding, e.g. the oral cavity via the heat transfer permeable outer layer 110 (not shown), via the heat collecting region 310 and the heat emitting region 311, to the internal heat buffer 250 at a temperature of its melting point and consisting of a mixture of solid and liquid regions, i.e. being a multi-state-mixture illustrated as “X” and “O”, respectively, according to one embodiment of the invention.

### DETAILED DESCRIPTION

Specific examples of the invention will now be described with reference to the accompanying drawings. This invention may, however, be embodied in many different forms and should not be construed as limited to the examples set forth herein, rather, these examples are provided so that this disclosure will be thorough and complete, and will fully convey the scope of the invention to those skilled in the art. The terminology used in the detailed description of the examples illustrated in the accompanying drawings is not intended to be limiting of the invention. In the drawings, like numbers refer to like elements.

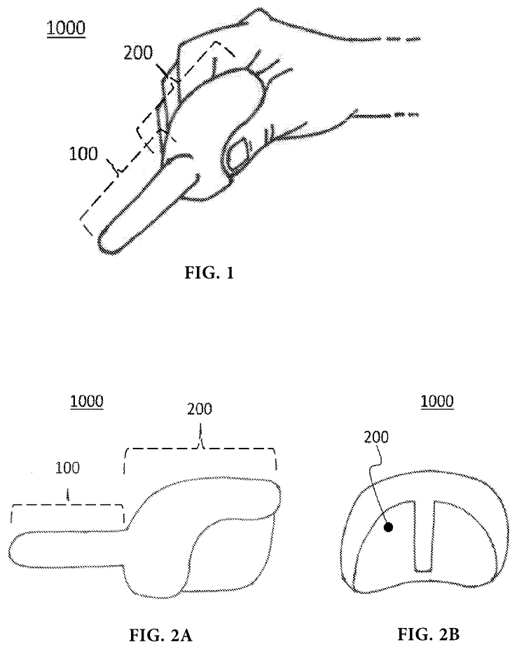

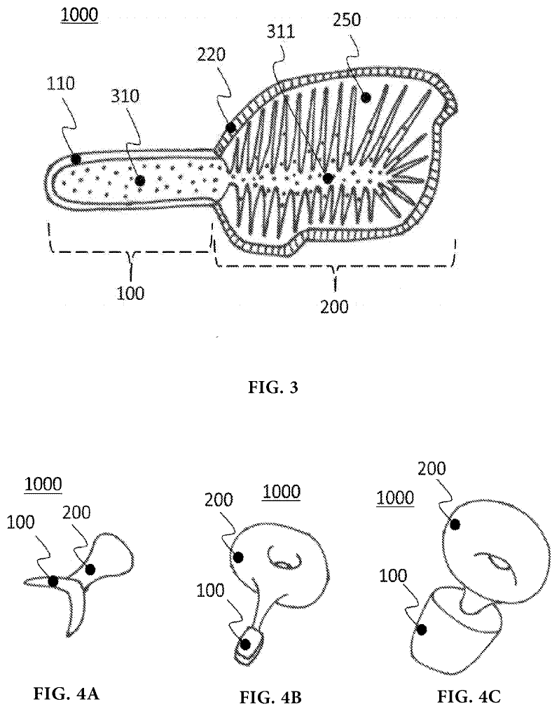

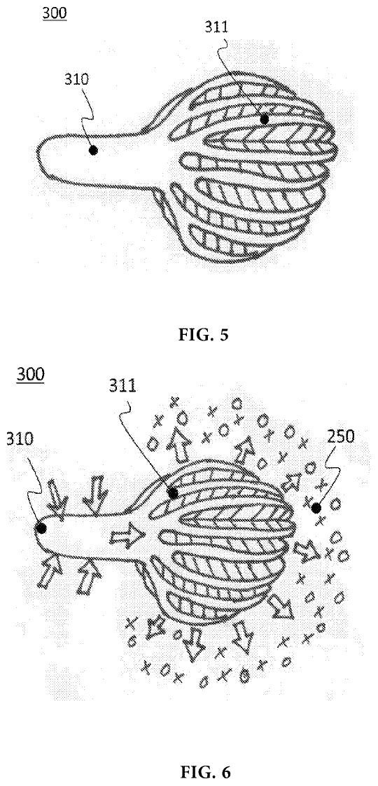

The oral cooling device 1000 of the invention may advantageously be used for cooling a singularity or plurality of separate or consecutive areas within the oral cavity, e.g. in order to ameliorate thirst. It essentially comprises a distal region 100 and a proximal region 200 (FIG. 1-4). The distal region 100, having a heat transfer permeable outer layer 110 on at least a part of its outer surface and a heat collecting region 310 of an internal heat transfer element 300 in its inner volume, is adapted for contacting and cooling one or several interior parts of the oral cavity (FIG. 3). The proximal region 200, having a heat transfer restrictive outer layer 220 preferably covering essentially its entire outer surface, a heat emitting region 311 of the internal heat transfer element 300 and an internal heat buffer 250 in its inner volume, is adapted for the user to hold or grip (FIGS. 1 and 3). Upon use, heat is transferred from the oral cavity to the internal heat buffer 250 via the heat collecting region 310 and the heat emitting region 311 of the internal heat transfer element 300 (FIG. 6). The proximal region 200 and the distal region 100 are spatially constricted relative each other, e.g. via the internal heat transfer element 300, whereby the user, e.g. a patient or care-giver, may control the placement of the effective cooling area of the distal region 100 in the oral cavity by holding and maneuvering the proximal region 200. The internal heat transfer element 300 may be a monolithic solid unit, a monolithic semisolid unit or a soft or semisoft bladder with thermally conductive filling, such as for example a gel-or semisolid filling comprising microscopic metal or metal oxide particles, or similar particles having a relatively high thermal conductivity. An example of other types of particles with appropriate thermal conductivity include various forms of carbon, as known in the art.

In a typical case of use, a caregiver, e.g. a nurse, may store a singularity or plurality of oral cooling devices 1000 at a temperature in the range from 20 to −25° C., such as 10to −20° C. or 5 to −10° C., to allow to come to a suitable temperature as known in the art, prior to use. The caregiver may then provide one cool oral cooling device 1000 to a patient suffering from thirst, instructing the same to hold the proximal region 200 and direct the cooling distal region 100 to target areas of choice within the oral cavity. The patient's thirst may then decrease by the type of dry, i.e. liquid free, cold which is provided by the cool oral cooling device 1000. After use, e.g. when the patient no longer is suffering from thirst or the internal heat buffer 250 has been heat saturated, i.e. no longer is cool enough to enable a desirable cooling effect, the caregiver may preferably clean the oral cooling device 1000, e.g. with ethanol, prior to re-cooling and repeating the cycle.

The heat transfer permeable outer layer 110 may be made of a hydrophobic non-permeable material which is medically acceptable, have low friction and high or acceptable heat permeability, as known in the art. Examples of suitable materials include, but is not limited to thermoplastic materials, e.g.

polyethylene or polypropylene, fluorinated organic polymers, e.g. Teflon, silicones and the like. The heat transfer permeable outer layer 110 may have a thickness equal to or less than 0.5 mm, such as equal to or less than 0.1 or 0.05 mm. It may cover 1 to 100 %, such as 30 to 90 % or 50 to 70 % of the outer surface of the distal region 100. Preferably it is located at regions of the distal region 100 expected to reach target areas in the oral cavity suitable for cooling to reach a desired thirst quenching effect. The remaining area of the distal region 100 may be made of any other suitable hydrophobic non-permeable material, optionally with relatively lower heat permeability, which is medically acceptable and has an acceptable low friction, as known in the art. The heat transfer permeable outer layer 110 may vary in thickness over the outer area of the distal region 100, such that relatively thin regions are located at regions where effective cooling is desired and relatively thick regions located at the other regions, as known in the art. The heat transfer permeable outer layer 110 may thus, advantageously from e.g. a manufacturing aspect, comprise the same type of material in its entire extension, but yet have regions with varying heat transfer capability, i.e. cooling capability, due to their thickness. The heat transfer permeable outer layer 110 may be in direct contact with the underlying heat collecting region 310, whereby advantageous effective heat transfer is achieved. The heat transfer permeable outer layer 110 may be in in indirect contact with the underlying heat collecting region 310 via e.g. a suitable gel as known in the art, whereby an advantageous exterior convenient softness of the distal region 100 may be experienced by the user, in particular in the case the internal heat transfer element 300 is a monolithic solid unit.

The heat transfer restrictive outer layer 220 is preferably covering essentially the entire outer surface of the proximal region 200, i.e. the part of the oral cooling device 1000 which is outside of the oral cavity when used, or at least the areas thereof being brought in direct contact with the users fingers or hand when used, in order to prevent undesired heat entrance from the surrounding environment. In the latter case, at least user contacting areas of the outer surface of the proximal region 200 may be covered by a heat transfer restrictive outer layer 220. The heat transfer restrictive outer layer 220 may be made of a suitable water resistant organic polymeric material having high resistance against heat transfer, e.g. a foamed soft material as known in the art. Preferably, it may have an outer surface that is adapted for facile cleaning, e.g. with ethanol.

The internal heat transfer element 300 may contain more than 1 weight-%, such as more than 10 or more than 50 weight-%, of a metal or oxide thereof selected from the group of metals consisting of aluminum, silver and copper and mixtures thereof, or any other suitable metal or metal oxide with sufficient heat transfer capability, as known in the art.

The internal heat buffer 250 may be a liquid, solid, semisolid, gel, or any mixture thereof, which may constitute more than 50 weight-%, such as more than 80, 90 or 95 weight-% water. It may consist of a mixture of different physical states, i.e. being a multi-state-mixture, at a specific temperature or within a specific temperature interval, e.g. a mixture of solid and liquid. At such a temperature or temperature interval, the transition of one state to another, e.g. from solid to liquid, can be achieved by absorption of energy without a rise in the overall temperature or a rise above the upper limit of such temperature interval. Hence, a significant amount of heat may be transferred from the oral cavity to the internal heat buffer 250 when being a multi-state-mixture, without a rise in temperature above a certain limit. Herein, the term “melting point” is to be understood as such a limit in this context. The operating cooling temperature of the oral cooling device 1000 may thus advantageously be held at a selected temperature, or withing a selected temperature interval, for a prolonged time when used by the utilization of an appropriately selected multi-state-mixture as internal heat buffer 250. Appropriate selection of the internal heat buffer 250 compositions may thus result in the formation of multi-state-mixtures at a desired temperature or temperature interval. Examples of suitable compositions constituting the internal heat buffer 250 with an ability to form multi-state-mixtures at a suitable temperature or within a suitable temperature interval include, for example, aqueous solutions of salts selected from the group of salts consisting of NaCl, NH₄Cl, (NH₄)₂SO₄, K₂SO₄, ZnSO₄, K₂HPO₄ and Na₂CO₃. Other suitable compositions are well understood by the skilled person.

According to one embodiment, the internal heat buffer 250 may be an aqueous salt solution, e.g. saline, solution having a melting point, i.e. forming a multi-state-mixture, in the range of −20 to −1° C., such as in the range of −15 to −5° C. Adapting the internal heat buffer 250 to be solid before use and to melt during use, will advantageously increase the heat buffering capacity at the temperature of the melting point as compared to the case when no phase transition from solid to liquid is occurring.

According to one embodiment, the internal heat transfer element 300 may be a monolithic unit made of aluminum, having a heat emitting region 311 comprising a plurality of extension adapted to display a maximized surface area, i.e. being heat sink shaped, as known in the art. Advantages of a monolithic heat transfer element 300 of aluminum includes a near optimal balance between price, relatively low weight and relatively high thermal conductivity.

According to one embodiment, the internal heat transfer element 300 may be a hollow watertight bladder, having a heat sink shaped outer area in its emitting region 311 and comprising a heat permeable gel, semisolid or liquid in its internal volume. The bladder may be made of rubber or any other suitable material known in the art for providing a soft or semisoft element with an internal filling that may change shape slightly as dependent on pressure applied onto it and having relatively high thermal conductivity. The heat permeable gel, semisolid or liquid may comprise more than 1 weight-%, such as more than 10 or more than 50 weight-%, of metal particles or metal oxide particles of a size in the range from nanoparticles to granules having a diameter of up to 3 mm, in mixture with a carrying-or lubricating agent that provides even spreading of the particles, as known in the art. Examples of heat permeable gels, semisolids or liquids include, but is not limited to, heat conductive pastes or gels comprising aluminum, silver, copper or carbon, organic-or silicon oils and the like.

According to one embodiment, the internal heat transfer element 300 may be a hollow watertight bladder, having a heat sink shaped outer area in its emitting region 311 and comprising a heat permeable gel, semisolid or liquid in its internal volume. The distal part thereof may be adapted to constitute the combined distal region 100 and the heat transfer permeable outer layer 110.

The bladder may thus advantageously grant exterior convenient softness to the distal region 100 as may be positively experienced by the user.

A list of further non-restrictive embodiments, classified by the integer numbers 1 to 8 and serving as examples of combinations of features that may constitute ways to carry out the present invention, follows below:
1. An oral cooling device 1000 for user controlled local cooling of the oral cavity, having a distal region 100 adapted for placement within the oral cavity, and a proximal region 200 adapted for being gripped by a hand or at least two fingers of the user, wherein
the distal region 100 is mechanically arranged in a spatially restricted relationship relative the proximal region 200 such that a user induced movement of the latter results in a corresponding movement of the former;
the outer surface of the distal region 100 is at least partly constituted by a heat transfer permeable outer layer 110 being in direct or indirect contact with a heat collecting region 310 of an internal heat transfer element 300 located in the inner volume of the distal region 100, for heat transfer from the oral cavity to the heat collecting region 310;
the outer surface of the proximal region 200 is essentially fully constituted by a heat transfer restrictive outer layer 220 enclosing an internal heat buffer 250 and a heat emitting region 311 of the internal heat transfer element 300 in the inner volume of the proximal region 200, for the prevention of heat transfer from the surrounding to the internal heat buffer 250 and to the heat emitting region 311;
the heat emitting region 311 and the internal heat buffer 250 are joined or in direct contact with each other and adapted to maximize heat transfer between each other; and
the heat collecting region 310 and the heat emitting region 311 are joined or in direct contact with each other to together form the internal heat transfer element 300 and adapted to maximize heat transfer between each other, for heat transfer from the oral cavity to the internal heat buffer 250.
2. An oral cooling device 1000 according to numbered embodiment 1, wherein the internal heat transfer element 300 contains more than 1 weight-%, such as more than 10 or more than 50 weight-%, of a metal selected from the group of metals consisting of aluminum and copper.
3. An oral cooling device 1000 according to any one of the preceding numbered embodiments, wherein the thickness of the heat transfer permeable outer layer 110 is equal to or less than 0.5 mm, such as equal to or less than 0.1 or 0.05 mm.
4. An oral cooling device 1000 according to any one of the preceding numbered embodiments, wherein the heat transfer restrictive outer layer 220 is made of a water-resistant organic polymer.
5. An oral cooling device 1000 according to any one of the preceding numbered embodiments, wherein the internal heat buffer 250 is an aqueous salt solution with a melting point in the range of −20 to −1° C.
6. Use of an oral cooling device 1000 according to any one of the preceding numbered embodiments for cooling an area within the human oral cavity.
7. Use of an oral cooling device 1000 according to claims 1 to 5 for the prevention, reduction or amelioration of thirst.
8. A method for cooling an area within the human oral cavity or for reduction or amelioration of thirst, comprising the consecutive steps (i) and (ii):
(i) cooling an oral cooling device 1000 according to claims 1 to 5 to a temperature being less than 30° C., such as less than 20, 10 or 0 ° C., to provide a cold oral cooling device 1000; and
(ii) cooling an area within the human oral cavity using the cold oral cooling device 1000 from step (i).

The scope of the invention is only limited by the appended patent claims.

More generally, those skilled in the art will readily appreciate that all parameters, dimensions, materials, and configurations described herein are meant to be exemplary and that the actual parameters, dimensions, materials, and/or configurations will depend upon the specific application or applications for which the teaching of the present invention is/are used.

1. An oral cooling device (1000) for user controlled local cooling of the oral cavity, having a distal region (100) adapted for placement within the oral cavity, and a proximal region (200) adapted for being gripped by a hand or at least two fingers of the user, wherein
the distal region (100) is mechanically arranged in a spatially restricted relationship relative the proximal region (200) such that a user induced movement of the latter results in a corresponding movement of the former;
the outer surface of the distal region (100) is at least partly constituted by a heat transfer permeable outer layer (110) being in direct or indirect contact with a heat collecting region (310) of an internal heat transfer element (300) located in the inner volume of the distal region (100), for heat transfer from the oral cavity to the heat collecting region (310);
the outer surface of the proximal region (200) is fully or partly covered by a heat transfer restrictive outer layer (220) enclosing an internal heat buffer (250) and a heat emitting region (311) of the internal heat transfer element (300) in the inner volume of the proximal region (200), for the prevention of heat transfer from the surrounding to the internal heat buffer (250) and to the heat emitting region (311);
the heat emitting region (311) and the internal heat buffer (250) are in direct contact with each other and adapted to maximize heat transfer between each other by the former being heat sink shaped;
the heat collecting region (310) and the heat emitting region (311) are joined with each other to together form the internal heat transfer element (300) and adapted to maximize heat transfer between each other, optionally by the internal heat transfer element (300) containing more than 1 weight-% of a metal or metal oxide, for heat transfer from the oral cavity to the internal heat buffer (250) ; and the internal heat buffer (250) is an aqueous salt solution with a melting point in the range of −20 to −1° C.

2. An oral cooling device (1000) according to claim 1, wherein the internal heat transfer element (300) contains more than 1 weight-%, such as more than 10 or more than 50 weight-%, of a metal or metal oxide selected from the group of metals consisting of aluminum, silver and copper.

3. An oral cooling device (1000) according to claim 1, wherein the heat collecting region (310) and the heat emitting region (311) together forms a water-tight bladder comprising a heat permeable gel, semisolid or liquid in its internal volume.

4. An oral cooling device (1000) according to claim 3, wherein the distal part of said bladder constitutes the distal region (100) and the heat transfer permeable outer layer

(110) thereof.

5. An oral cooling device (1000) according to claim 1, wherein the thickness of the heat transfer permeable outer layer (110) is equal to or less than 0.5 mm, such as equal to or less than 0.1 or 0.05 mm.

6. An oral cooling device (1000) according to claim 1, wherein the heat transfer restrictive outer layer (220) is made of a water-resistant organic polymer.

7. Use of an oral cooling device (1000) according to claim 1 for cooling an area within the human oral cavity.

8. Use according to claim 7, wherein the internal heat buffer 250 being a multi-state-mixture comprising local regions being in one physical state, e.g. in a solid state, and other regions being in another physical state, e.g. in a liquid state.

9. A method for cooling an area within the human oral cavity or for reduction or amelioration of thirst, comprising the consecutive steps (i) and (ii):
(i) cooling an oral cooling device (1000) according to claim 1 to a temperature being less than 0° C., to provide a cold oral cooling device (1000); and
(ii) cooling an area within the human oral cavity using the cold oral cooling device (1000) from step (i).
